(19) United States
(12) Patent Application Publication
(10) Pub. No.: US 2005/0267169 A1
(43) Pub. Date: Dec. 1, 2005

(54) Watersoluble prodrugs of propofol

(76) Inventors: Michele Orlando, Giessen (DE); Jurgen Hemberger, Aschaffenburg (DE); Giuseppe Trapani, Molfetta (IT); Gaetano Liso, Bari (IT); Cosimo Altomare, Molfetta (IT); Andrea Latrofa, Noicattaro (IT); Giovanni Biggio, Cagliari (IT); Mariangela Serra, Cagliari (IT); Enrico Sanna, Assemini (IT); Valentino Laquintana, Monopoli (IT)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/100,746

(22) Filed: Apr. 7, 2005

(30) Foreign Application Priority Data
Apr. 8, 2003 (WO) PCT/EP03/03642
Oct. 8, 2002 (DE) DE 202 15 415.7

(51) Int. Cl. A61K 31/455; A61K 31/401

(52) U.S. Cl. 514/355; 514/423; 546/315; 548/530

(57) ABSTRACT

The present invention relates to propofol derivatives comprising a cyclic or linear amino acid, or a poly- or (oligo)saccharide moiety, a process for preparing said derivatives, a method for anesthetizing a mammal as well as a method for treating convulsions, migraine or related diseases, or for the inhibition of free radicals in a mammal to which said compounds are administered. Furthermore, the present invention relates to said compounds for use as a medicament and the use of said compounds for the preparation of a medicament for anesthetizing a mammal or for treating convulsions, migraine or related diseases, or for inhibition of free radicals in a mammal.

### CROSS REFERENCE TO RELATED APPLICATIONS

This application is a Continuation of PCT Patent Application No. PCT/EP2003/003642, filed Apr. 8, 2003, which claims priority to German Patent Application No. 202 15 415.7, filed Oct. 8, 2002, all of which are hereby incorporated by reference in their entirely herein.

### FIELD OF THE INVENTION

The present invention relates to propofol derivatives comprising a cyclic or linear amino acid, or a poly- or (oligo)saccharide moiety, a process for preparing said derivatives, a method for anesthetizing a mammal as well as a method for treating convulsions, migraine or related diseases, or for the inhibition of free radicals in a mammal to which said compounds are administered. Furthermore, the present invention relates to said compounds for use as a medicament and the use of said compounds for the preparation of a medicament for anesthetizing a mammal or for treating convulsions, migraine or related diseases, or for inhibition of free radicals in a mammal.

### BACKGROUND OF THE INVENTION

Propofol (2,6-diisopropylphenol, see compound 1 of FIG. 1) is an important intravenous agent in the practice of anesthesia. Due to its very low solubility in water, propofol was initially formulated as a 1% w/v solution in the presence of Cremophor EL (a solubilizing surfactant), but the anaphylactic reactions associated with its administration have led to a search for alternative formulations (Trapani G, Altomare C, Sanna E, Biggio G, Liso G., 2000; Propofol in anesthesia, Mechanism of action, structure-activity relationships, and drug delivery; Curr. Med. Chem. 7: 249-271; Franks N P, Lieb W R., 1994; Molecular and cellular mechanisms of general anaesthesia; Nature (Lond). 367: 607-614.). Presently, propofol is formulated in as an oil-in-water emulsion (1% w/v) of soya bean oil, glycerol and purified egg phosphatide (Diprivan®, Zeneca UK). Intravenous (i.v.) injection of Diprivan® produces hypnosis rapidly (usually within 40 sec) and smoothly with minimal excitation, but pain at the site of injection is a major adverse effect (Prankerd R D, Stella V J., 1990; Use of oil-in-water emulsions as a vehicle for parenteral drug administration; J. Parent. Sci. Technol. 44: 139-149.). As a lipid-based emulsion, it suffers from a number of limitations, such as poor physical stability, potential for embolism, and need for strictly aseptic handling (Bennett S N, Mc Neil M M, Bland L A, Arduino M J, Villarino M E, Perrotta D M, 1995; Postoperative infections traced to contamination of an intravenous anesthetic, propofol; New England Journal of Medicine 333: 147-154.). Moreover, particular care is required in patients with disorders of fat metabolism (Dollery C. (ed.), 1991; Propofol. In Therapeutics Drugs, Churchill Livingstone, London, Vol 2 pp. 269-271), and the material of the tubes used for infusing the emulsion must be carefully selected.

To avoid these drawbacks, safe alternative dosage forms, in particular aqueous formulations are needed. Approaches in this direction include the complexation of propofol with hydroxypropyl-β-cyclodextrin (Brewster, M. 1991; Parenteral safety and applications of 2-hydroxypropyl-β-cyclodextrin; In Duchêne D, editor, New Trends in Cyclodextrins and Derivatives, Paris: Editions de Santé, pp. 313-350; Trapani G, Lopedota A, Franco M, Latrofa A, Liso G, 1996; Effect of 2-hydroxypropyl-β-cyclodextrin on the aqueous solubility of the anesthetic agent propofol (2,6-diisopropylphenol); Int. J. Pharm. 139: 215-218; Trapani G, Latrofa A, Franco M, Lopedota A, Sanna E, Liso G. 1998. Inclusion complexation of propofol with 2-hydroxypropyl-β-cyclodextrin. Physicochemical, nuclear magnetic resonance spectroscopic studies, and anesthetic properties in rat. J. Pharm. Sci. 87: 514-518.) and chemical delivery systems. The main objectives of these approaches are to increase the hydrosolubility of propofol, improve patient acceptance, e.g. reduce pain at the site of injection, and a decrease in side-effects as well as prolonged action (Pop E, Anderson W, Prokai-Tatrai K, Vlasak J, Brewster M E, Bodor N., 1992; Syntheses and preliminary pharmacological evaluation of some chemical delivery systems of 2,6-diisopropylphenol (propofol); Med. Chem. Res. 2: 16-21.). Water-soluble prodrugs of propofol have also been prepared as suitable formulations for parenteral administration (Morimoto B H, Barker P L; Preparation of phosphocholine linked prodrug-derivatives. WO 00 48572; Stella V J, Zygmunt J J, Geog I G, Safadi M S; Water-soluble prodrugs of hindered alcohols or phenols, WO 00 08033; Sagara Y, Hendler S, Khon-Reiter S, Gillenwater G, Carlo D, Schubert D, Chang J, 1999; Propofol hemisuccinate protects neuronal cells from oxidative injury; J. Neurochem. 73: 2524-2530; Hendler S S, Sanchez R A, Zielinski J., Water-soluble prodrugs of propofol; WO 99 58555.)

α-Aminoacid ester derivatives of propofol (see compounds 2a-c of FIG. 1) (Trapani G, Latrofa A, Franco M, Lopedota A, Maciocco E, Liso G., 1998; Water-soluble salts of amino acid esters of the anesthetic agent propofol; Int. J. Pharm. 175: 195-204.) were investigated as prodrugs, which demonstrated good aqueous solubility and stability. But the resistance of these compounds against hydrolytic activation in plasma and brain homogenate is much too high for them to actually be considered true prodrugs. Interestingly, some of them were found to interact with the subtype A of the γ-aminobutyric acid (GABA_{A}) receptor, a major target mediating the pharmacological actions of propofol and other general anesthetics (Trapani G, Altomare C, Sanna E, Biggio G, Liso G, 2000; Propofol in anesthesia. Mechanism of action, structure-activity relationships, and drug delivery; Curr. Med. Chem. 7: 249-271; Franks N P, Lieb W R., 1994; Molecular and cellular mechanisms of general anaesthesia; Nature (Lond). 367: 607-614.). Nevertheless, due to their binding affinity to the GABA_{A} receptors, similar to that of parent propofol, some of them were suggested as promising candidates for in vivo pharmacological evaluation.

In summary, there is still a need for stable and water soluble propofol derivatives, that are capable of hydrolytic activation under physiological conditions. Specifically, there is a need for stable and water soluble prodrugs of propofol that are readily metabolized to release propofol in vivo.

### DISCLOUSURE OF THE INVENTION

The present invention provides in one aspect propofol derivatives having the formula:

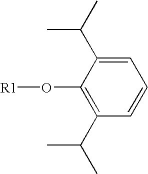

wherein R1 is a cyclic or linear amino acid and wherein the propofol derivative is present in the form of a free base or salt. Said amino acid may be present in the form of their diastereomers or enantiomers. Optionally, the amino acid can be further substituted.

According to the present invention the term “amino acid” means any artificial or naturally occurring amino acid characterized by the presence of an amino or imino group and a carboxy group. The term encompasses cyclic and non-cyclic compounds, wherein the cyclic compound may be aromatic or alicyclic. Preferably, the amino acid is a naturally occuring amino acid or a derivative thereof. Preferably, the amino acid is an alpha-, beta-, gamma-, delta- or epsilon-amino acid.

Preferably, the amino acid is C-terminally linked to propofol.

It is preferred that the salts include chloride, sulphate, (hemi)tartrate, (hemi)succinate, (hemi)malate, acetate, lactate and similar anions.

According to a preferred embodiment of the present invention the propofol derivatives have the formula:

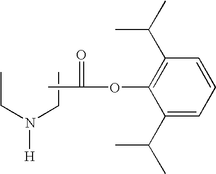

wherein the heterocyclic group comprises 4 to 5 methylene groups and wherein the heterocyclic group is optionally further substituted. In a preferred embodiment, R1 is an oligoamino acid having from 2 to 5 amino acid moieties.

Preferably, R1 does not comprise a tertiary nitrogen. More preferably, R1 does not comprise a tertiary nitrogen and the compounds of the present invention comprise the above mentioned heterocyclic group, which in turn comprises 4 to 5 methylene groups and wherein the heterocyclic group is optionally further substituted.

It is further preferred that the compounds may be subject to rapid cleavage by esterases.

More preferably, R1 is selected from proline and the three positional isomers of piperidine i.e., pipecolinic, nipecotic, and isonipecotic acid.

It is also more preferred that R1 is selected from the group consisting of tyrosine, tryptophan, phenylalanine or histidine.

The aromatic ring may also be further substituted to create condensed or fused aromatic compounds of the naphthaline, anthracene or phenanthrene-type. It is however required that said compounds are essentially water-soluble.

More preferably, said compounds are selected from α-proline, α-pipecolinic acid, β-nipecotic acid and γ-isonipecotic acid.

For migraine application the preferred compounds are those which act as depot form i.e. are cleaved more slowly.

In a further preferred embodiment of the present invention, said compounds are selected from α-proline, α-pipecolinic acid, or β-nipecotic acid, preferably from α-proline or α-pipecolinic acid, and most preferably said compound is α-proline.

The amino acid compound may also be a linear amino acid. Preferably, the amino acid is selected from glycine, alanine, valine, leucine, isoleucine, glutamine, glutamic acid, asparagine, aspartic acid, cysteine, methionine, serine, or threonine.

The skilled person is aware that the amino acid component of propofol derivatives according to the invention is of a basic nature due to the secondary nitrogen atom within the cyclic structure. Therefore, the compounds of the present invention tend to form salts. Preferred salts of the propofol derivatives of the present invention comprise hydrogen ion and any suitable pharmaceutically acceptable counterion, preferably selected from the group of chloride, sulphate, (hemi)tartrate, (hemi)succinate, (hemi)malate, acetate, lactate and similar anions.

According to another aspect of the invention the propofol derivatives have the formula:

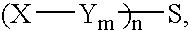

wherein X has the formula:

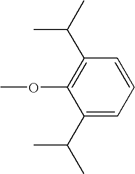

Y is a bifunctional linker,

S is a poly- or oligosaccharide moiety,

n is equal or less than the number of the terminal saccharide units in the poly- or oligosaccharide S, and

m is, independent of n, 0 or 1.

Preferably, in the propofol derivatives of the present invention, m=0 and propofol and S are linked to each other by an ester bond consisting of an oxygen of X and a terminal carbonyl derivative of S.

Also preferred are propofol derivatives of the present invention, wherein m=1 and propofol and S are linked to each other by means of a bifunctional linker Y, said bifunctional linker Y preferably being linked to X by an ester, carbonate or carbamate bond and being linked to S by an amide, amine, secondary amine, imine, ether, ester, thioester, carbonate, carbamate, urea or disulfide bond.

In a preferred embodiment, the saccharide S is an oligosaccharide comprising at most 1 to 20, preferably 1 to 10, more preferably 2 to 7 saccharide units.

The term “oligosaccharide” as used herein is defined as encompassing 1 to 20 saccharides. It is emphasized that mono-, di-, and trisaccharides are specifically included in the definition of oligosaccharides.

It was surprisingly found that insoluble propofol does not require large hydrophilic polymers to produce the desired hydrophilicity in a conjugate. Unexpectedly, 1 to 20 saccharide units are found to be sufficient. Conjugates according to the present invention can easily be produced with the homogeneity that is necessary for a predictable and desirable pharmacokinetic profile as well as enhanced biocompatibility.

In a further preferred embodiment, the saccharide S is a polysaccharide consisting of more than 20 saccharide units, preferably 20 to 100, more preferably of 20 to 50 saccharide units.

The poly- or oligosaccharide S may be linear or branched and the saccharide monomers within the polysaccharide are linked to each other by α- or β(1-2), (1-4), or (1-6) bonds.

Preferably the polysaccharide is branched (e.g. HES, hydroxy ethy starch), and more preferably the polysaccharide is branched and the saccharide units within the polysaccharide are linked by α- or β(1-4) bonds and α- or β(1-6) bonds at the branching points. In the most preferred embodiment, the polysaccharide is branched and the saccharide units within the polysaccharide are linked by α(1-4) bonds and by α(1-6) bonds at the branching points.

In an alternative preferred embodiment, the oligosaccharide is linear, and more preferably the oligosaccharide is linear and the saccharide units within the oligosaccharide are linked by α- or β(1-4) bonds. In the most preferred embodiment, the oligosaccharide is linear and the saccharide units within the oligosaccharide are linked by α(1-4) bonds.

In a preferred embodiment, poly- or oligosaccharide S comprises at least one terminal aldose saccharide unit(s) having a free reducing end. More preferably, oligosaccharide S comprises at least one terminal saccharide unit of S that is or are derived from an aldose monosaccharide comprising a free aldehyde group.

The term “terminal saccharide” as used herein refers to a saccharide unit by itself, in a poly- or oligosaccharide that is only linked to none or one further saccharide unit.

When using smaller oligosaccharides according to this invention yet another important advantage is the possibility to solubilize a much higher amount of the pharmaceutically active substance without yielding highly viscous solutions, that are generally observed for polymer-conjugated small molecules at high concentrations. For example, a trisaccharide (e.g., maltotrionic acid) conjugated drug will achieve an almost 100 times higher concentration compared to the same drug coupled to hydroxyethyl starch with 50 kD molar mass before reaching an acceptable limit of viscosity. Therefore, higher concentrations of the therapeutic component can be reached much easier with the conjugates according to this invention. As a consequence, conjugates of the invention are not only easier to handle for galenic formulations (e.g. reduced side effects such as, e.g., reduced deposition of the conjugate at the site of administration and reduced accumulation in undesired locations in a body) and clinical applications but also allow a higher therapeutic dosage in comparison to HES or PEG conjugates. For a review on viscosity and physiology see J. D. Bronzine, The Biomedical Engineering Handbook, CRC Press, USA, Salem 1995.

In a preferred embodiment the viscosity of the propofol derivatives according to the invention is 1-100 mPasc, preferably 1-20 mpasc, more preferably 1-7 mPasc.

In a preferred embodiment the molar ratio of propofol to S is in the range of 10:1 to 1:1, preferably in the range of 5:1 to 1:1, more preferably the ratio of X to S is 1:1.

The poly- or oligosaccharide S may comprise one or more physiologically acceptable saccharide unit(s). Preferably, S comprises one or more of the poly- or oligosaccharide unit(s) selected from the group consisting of:
a) monosaccharides, preferably: ribose, arabinose, xylose, lyxose, allose, altrose, glucose, mannose, gulose, idose, galactose, talose, fucose;
b) disaccharides, preferably lactose, maltose, isomaltose, cellobiose, gentiobiose, melibiose, primeverose, rutinose;
c) disaccharide homologues, preferably maltotriose, isomaltotriose, maltotetraose, isomaltotetraose, maltopentaose, maltohexaose, maltoheptaose, lactotriose, lactotetraose;
d) uronic acids, preferably glucuronic acid, galacturonic acid;
e) branched oligosaccharides, preferably panose, isopanose,
f) amino monosaccharides, preferably galactosamine, glucosamine, mannosamine, fucosamine, quinovosamine, neuraminic acid, muramic acid;, lactosediamine, acosamine, bacillosamine, daunosamine, desosamine, forosamine, garosamine, kanosamine, kansosamine, mycaminose, mycosamine, perosamine, pneumosamine, purpurosamine, rhodosamine;
g) modified saccharides, preferably abequose, amicetose, arcanose, ascarylose, boivinose, chacotriose, chalcose, cladinose, colitose, cymarose,2-deoxyribose, 2-deoxyglucose, diginose, digitalose, digitoxose, evalose, evernitrose, hamamelose, manninotriose, melibiose, mycarose, mycinose, nigerose, noviose, oleandrose, paratose, rhodinose, rutinose, sarmentose, sedoheptulose, solatriose, sophorose, streptose, turanose, tyvelose.

More preferably, S comprises one or more of the poly- or oligosaccharide unit(s) selected from the group consisting of glucosamine, galactosamine, glucuronic acid, galacturonic acid, lactose, lactotetraose, maltose, maltotriose, maltotetraose, isomaltose, isomaltotriose, Isomaltotetraose, and neuraminic acid.

In a preferred embodiment, the bifunctional linker Y is a linker that is non-toxic and physiologically acceptable. More preferably, the linker Y comprises a linear or branched aliphatic chain, preferably an aliphatic chain of 1 to 20, more preferably 1 to 12, most preferably 2 to 6 carbons.

In a most preferred embodiment, the bifunctional linker Y is
—HN—(CH₂)ₓ—NH—CO—(CH₂)_{y}—CO—,
wherein X=0 to 10, preferably X=0 to 4, and Y=0 to 5, preferably Y=1 or 2.

In a further preferred embodiment, S is a monosaccharide, disaccharide, oligosaccharide or polysaccharide comprising at least one moiety selected from allose, altrose, glucose, mannose, gulose, idose, galactose, talose, sucrose, lactose, maltose, isomaltose, cellobiose, maltobionic acid, and lactobionic acid.

Also preferred is that S is maltotrionic acid, lactobionic acid or hydroxyethyl starch.

Particularly preferred, is that S comprises at least 2 hydroxyethyl glucose units, wherein optionally the hydroxy ethyl glucose units may be substituted. Reference is made to German patent application DE 10209822.0, the disclosure of which, in particular with respect to the glycosylation pattern of hydroxyethyl starch is incorporated herewith.

The linker group may be any linker group known in the art provided that the produced compound is still sufficiently water-soluble. Linker of the hydrazine or glutaric acid type and homologs thereof are preferred.

For the skilled person the preparation of the conjugates of the present invention is within his average skill and merely requires routine experimentation and optimization of standard synthesis strategies that are abundantly available in the prior art. Numerous non-degrading and selective strategies are available for linking amine, alcohol, and thiol functional groups with aldehyde, carboxylic acid or activated carboxylic acid functional groups. If component X and/or S lack the desired functional group it may be introduced by chemical derivatization of existing functional groups, the addition of suitable functional groups, or the addition of suitable functional linker molecules.

In a further aspect, the present invention relates to a process for preparing propofol derivatives according to the present invention, comprising the steps of:
a) coupling propofol with one or more terminal aldehyde group(s) of a poly- or oligosaccharide S, or
b) coupling propofol with one or more terminal carboxylic group(s) of a poly- or oligosaccharide S, or
c) coupling propofol with one or more activated terminal carboxylic group(s) of a poly- or oligosaccharide S.

An activated carboxylic group in that respect means any carboxylic group derivative that displays a higher reactivity towards a nucleophile than the original carboxylis group (for an example see below).

The functional group involved in the coupling reaction of the process of the present invention can be the aldehyde functional group of one or more terminal saccharide units in the oligosaccharide S. This aldehyde functional group can be used as such or be further chemically modified.

In a preferred embodiment, the process of the invention further comprises a step b′) or c′) prior to step b) or c), respectively, wherein one or more tenminal aldehyde group(s) of a poly- or oligosaccharide S precursor are selectively oxidized to produce the poly- or oligosaccharide S.

Preferred oxidation procedures for selectively oxidizing terminal aldehyde group(s) of oligosaccharide S are those using
(i) halogen, preferably I₂, Br₂, in alkaline solution, or
(ii) metal ions, preferably Cu⁺⁺ or Ag⁺, in alkaline solution, or
(iii) by electrochemical oxidation.

The resulting carboxylic acid can be used in the coupling reaction to yield an ester with propofol.

The carboxyl group can be used as such or after a previous activation step, that yields an activated carboxylic acid group, such as, e.g. a lactone, an active ester, a symmetric anhydride, a mixed anhydride, a halogenide of a carboxylic acid or any other activated form of a carboxylic group that is suitable to produce the desired ester bond.

Preferred examples of activated carboxylic acids are selected from the group consisting of a lactone, an anhydride, a mixed anhydride, and a halogenide of a carboxylic acid.

More preferred active esters are esters of p-nitrophenol; 2,4,6-trinitrophenol; p-chlorophenol; 2,4,6-trichlorophenol; pentachlorophenol; p-fluorophenol; 2,4,6-trifluorophenol; pentafluorophenol; N-hydroxybenzotriazole; N-hydroxysuccinimide;

Active ester can, for example, be formed by using one of the follwing reagents: N-hydroxy succinimide, N-hydroxy phthalimide, thiophenol, p-nitrophenol, o,p-dinitrophenol, trichlorophenol, trifluorophenol, pentachlorophenol, pentafluorophenol, 1-hydroxy-1H-benzotriazole (HOBt), HOOBt, HNSA, 2-hydroxy pyridine, 3-hydroxy pyridine, 3,4-dihydro-4-oxobenzotriazin-3-ol, 4-hydroxy-2,5-diphenyl-3(2H)-thiophenone-1,1-dioxide, 3-phenyl-1-(p-nitrophenyl)-2-pyrazolin-5-one), [1-benzotriazolyl-N-oxy-tris(dimethylamino)-phosphoniumhexa-fluorophosphate] (BOP), [1-benzotriazolyloxytripyrrolidinophosphonium-hexafluoro-phosphate (PyBOP), [O-(benzotriazol-1-yl)-N,N,N′,N′-tetramethyluroniumhexa-fluorophosphate (HBTU), [O-(benzotriazol-1-yl)-N,N,N′,N′-tetramethyluronium-tetrafluoroborate (TBTU), [O-(benzotriazol-1-yl)-N,N,N′,N′-bis(pentamethylen)uronium-hexafluorophosphate, [O-(benzotriazol-1-yl)-N,N,N′,N′-bis(tetramethylen)uronium-hexafluorophosphate, carbonyidiimidazole (CDI), carbodiimides, examples are 1-(3-dimethylaminopropyl)-3-ethylcarbodiimide (EDC), dicyclohexylcarbodiimide (DCC), diisopropylcarbodiimide (DIPC).

Preferred is a process of the invention, wherein in step c) the one or more activated terminal carboxylic group(s) of a poly- or oligosaccharide S are activated carboxylic acids selected from the group consisting of a lactone, an anhydride, a mixed anhydride, and a halogenide of a carboxylic acid.

Preferably, the process of the invention is one, wherein in step c) the one or more activated terminal carboxylic group(s) of a poly- or oligosaccharide S is (are) a lactone functional group(s). Preferably such a lactone group results from the oxidation of a terminal aldehyde group of an aldose. More preferably, the oxidation is performed with I₂ in the presence of NaOH, yielding a carboxylic acid intermediate functional group that is transformed into a lactone by water elimination.

The oligosaccharide lactone derivative is sufficienty active to react with a primary alcohol function. Typically, the presence of activators, e.g., carbodiimides, is necessary.

Due to the low stability in water of such lactones and due to the low water solubility of the pharmaceutically active component the reaction is preferably performed in presence of a suitable organic solvent.

Preferred organic solvents are polar non-protic ones (DMF, DMSO, N-methylpyrrolidone and the like) or lower alcohols (C₁₋₁₀, e.g. MeOH, EtOH, n-PrOH, i-PrOH, n-butanol, i-butanol, tert-butanol, glycol, glycerol etc.). In specific cases it may also be of advantage to perform the reaction in heterogeneous phase, e.g. in a liquid heterogenous phase such as a dispersion.

Another way of transforming and linking functional groups according to the invention is by means of introducing a bifunctional linker that comprises at least two functional groups that are compatible with the selected propofol and S.

In a further aspect the present invention relates to a process for preparing compounds according to the invention, comprising the steps of:
a) coupling a suitable bifunctional linker group(s) Y to propofol , and
b) coupling the product(s) of step a) with one or more terminal aldehyde, carboxylic acid, or activated carboxylic group(s) of a poly- or oligosaccharide S, or
a′) coupling a suitable bifunctional linker group(s) to one or more terminal aldehyde, carboxylic acid, or activated carboxylic group(s) of a saccharide S, and
b′) coupling the product(s) of step a) with one or more propofol.

When an imine bond is formed between the bifunctional linker group Y and the poly- or oligosaccharide S, it is preferably further reduced to a secondary amine.

It is especially preferred that the imine is reduced by NaBH₃CN at a pH values of 6-7.

It is also preferred that in step b) or step a′) the one or more activated terminal carboxylic group(s) of poly- or oligosaccharide S selected from the group consisting of a lactone, an anhydride, a mixed anhydride, and a halogenide of a carboxylic acid.

Preferably, the activated carboxylic acid is a lactone. The poly- or oligosaccharide lactone derivative is sufficiently active to react with a primary amino function of the bifunctional linker Y. In contrast to the normal conditions that are used for similar coupling reactions, that usually require the presence of activators, e.g., carbodiimides, it was surprisingly found that the reaction also proceeds readily with high chemical yields without an activator. This is a substantial advantage in that additional purification steps that are necessary for separating the activator and its by-products are redundant.

Preferably, the coupling of a lactone poly- or oligosaccharide derivative and one or more bifunctional linkers Y is performed in the absence of an activator.

In a preferred embodiment, the lactone is coupled in non-protic solvents mentioned before.

Suitable linker molecules are those that have at one end any reactive functional group that reacts with the propofol and at the other end any reactive functional group that is able to react with a poly- or oligosaccharide S. Preferably, said bifunctional linker reacts with an alcohol of propofol and an amine, alcohol, thiol, aldehyde, carboxylic acid, or activated carboxylic acid of S.

In a preferred embodiment, the the bifunctional linker used in the process of the present invention is preferably non-toxic and physiologically acceptable. More preferably, the bifunctional linker comprises a linear or branched aliphatic chain, preferably an aliphatic chain of 1 to 20, more preferably 1 to 12, most preferably 2 to 6 carbon atoms.

It is more preferred that the bifunctional linker is a linker that has an amino functional group on one side to be coupled to the terminal saccharide moiety of S and an activated carboxylic function at the side to be coupled to propofol.

In a most preferred embodiment, the bifunctional linker is
—HN—(CH₂)ₓ—NH—CO—(CH₂)_{y}—CO—,
wherein X=0 to 10, preferably X=0, and Y=0 to 5, preferably Y=1 or 2.

In one specific embodiment of the present invention, water-soluble derivatives of propofol are preferably prepared by esterifying the drug with cyclic amino acids, preferably with four specific cyclic aminoacids (compounds 6a-d, see FIG. 1), namely proline and the three positional isomers of piperidine carboxylic acids (i.e., pipecolinic, nipecotic, and isonipecotic acids).

In another specific embodiment, water-soluble derivatives of profolol are obtained by esterifying a saccharide either directly or indirectly via linker groups with propofol. Examples for synthesis are given in the detailed description below.

Their properties such as e.g. solubility, lipophilicity, stability in aqueous solutions, and/or susceptibility to enzymatic hydrolysis in animal plasma and liver, and their ability to interact with GABA_{A} receptors make them excellent candidate substances for promoting anesthesia and for treating convulsions, migraine or related diseases and for inhibiting free radicals.

Three of the preferred amino acids that were esterified with propofol (with the exception of pipecolinic acid (3b)) are pharmacologically active on their own. (S)-Proline is an inibitory amino acid, (R)-nipecotic acid is an inhibitor of GABA uptake, and isonipecotic acid is a specific GABA_{A} agonist (Krogsgaard-Larsen P., Frolund B., Kristiansen U., Frydenvang K., Ebert B, 1977; GABA_{A} and GABA_{B} Receptor Agonists, Partial Agonists, Antagonists, and Modulators: Design and Therapeutic Prospects. 5: 355-384). Thus, with the exclusion of propofol pipecolinate (6b), the preferred ester derivatives 6a, 6c and 6d may be considered rather “dual prodrugs”, that are converted in vivo into two active molecules. Except proline, taken in its natural enantiomeric form (S), the other chiral amino acids (i.e., pipecolinic and nipecotic acids) were used in synthesis as racemates. The influence of their steric confomiation was not further investigated at this point.

Prolinate derivative 6a is particularly well suited as a water-soluble prodrug. Said compound protects animals against pentylenetetrazole-induced convulsions, and induces an anesthetic action in a short time of a duration that is comparable with that of the marketed propofol emulsion Diprivan®. Its high solubility and stability in water at physiological pH allow to prepare freeze-dried formulations for parenteral administration. The prolinate derivative 6a is a most preferred embodiment of the present invention.

In a preferred embodiment, the present invention relates to a freeze-dried pharmaceutical composition comprising at least one of the compounds of the present invention, more preferably comprising an α-proline propofol ester.

The susceptibility of the preferred proline ester 6a to enzymatic cleavage by ester hydrolases in plasma and liver affords conversion in vivo to the parent drug. Consequently, a 17 mg/mL aqueous solution of proline ester 6a, is equivalent to the commercial oil-in-water emulsion Diprivan® containing 10 mg/mL of propofol.

Prolinate 6a as well as piperidine-2-carboxylate 6b bind as such, i.e. as intact non-hydrolyzed molecules, to the propofol binding site of GABA_{A}. receptors, with IC₅₀ values of 30-40 μM (one log unit lower than propofol).

The non-sugar propofol esters of the present invention have demonstrated their pharmacological potential in an in vitro [35S]TBPS binding assay using rat brain and electrophysiological studies using Xenopus oocytes. Moreover, said compounds have demonstrated a pharmacologically effective anticonvulsant and anesthetic activity in vivo.

In general, the compounds of the present invention demonstrate high solubility and stability in aqueous solutions and also in physiological media in vitro. Non-sugar propofol esters are readily hydrolyzed in plasma and liver esterase solutions, many of them even quantitatively within a few minutes.

The compounds of the present invention are also efficacious in vivo. Because said compounds readily hydrolized under physiological conditions and release propofol, they are excellent prodrugs for propofol action.

Therefore, the present invention is also directed at a method for anesthetizing a mammal or a method of treating and/or preventing convulsions, migraine or related diseases or for inhibiting free radicals in a mammal, wherein a therapeutically effective amount of a compound according to the invention is administered to said mammal.

The term “preventing” as used herein is to be understood to refer to all processes, wherein the onset of a disease is delayed or eliminated.

The term “treating” is intended to refer to all processes, wherein there may be a slowing, interrupting, arresting, or stopping of the progression of a convulsion or convulsions, but does not necessarily indicate a total elimination of all symptoms.

The term “anesthetizing” as used herein is to be understood in the context of the pharmaceutical action of the parent compound propofol.

As used herein, the term “mammal” refers to a warm blooded animal. It is understood that guinea pigs, dogs, cats, rats, mice, horses, cattle, sheep, monkeys, chimpanzees and humans are examples of mammals and within the scope of the meaning of the term. Humans are preferred.

In effecting treatment of a mammal in need of anesthetic treatment or suffering from convulsion, the compounds disclosed by the present invention for said purpose can be administered in any form or mode which makes the therapeutic compound bioavailable in an effective amount, including oral or parenteral routes. For example, products of the present invention can be administered intraperitoneally, intranasally, buccally, topically, orally, subcutaneously, intramuscularly, intravenously, transdermally, rectally, and the like.

Parenteral administration of the compounds of the present invention is preferred.

One skilled in the art of preparing formulations can readily select the proper form and mode of administration depending upon the particular characteristics of the product selected, the disease or condition to be treated, the stage of the disease or condition, and other relevant circumstances. (Remington's Pharmaceutical Sciences, Mack Publishing Co. (1990)). The products of the present invention can be administered alone or in the form of a pharmaceutical preparation in combination with pharmaceutically acceptable carriers or excipients, the proportion and nature of which are determined by the solubility and chemical properties of the product selected, the chosen route of administration, and standard pharmaceutical practice. For oral application suitable preparations are in the form of tablets, pills, capsules, powders, lozenges, sachets, cachets, suspensions, emulsions, solutions, drops, juices, syrups, while for parenteral, topical and inhalative application suitable forms are solutions, suspensions, easily reconstitutable dry preparations as well as sprays. Compounds according to the invention in a sustained-release substance, in dissolved form or in a plaster, optionally with the addition of agents promoting penetration of the skin, are suitable percutaneous application preparations. The products of the present invention, while effective themselves, may be formulated and administered in the form of their pharmaceutically acceptable salts, such as acid addition salts or base addition salts, for purposes of stability, modulation of hydrophobicity, increased solubility, and the like.

The amount of active agent to be administered to the patient depends on the patient's weight, on the type of application, symptoms and the severity of the illness. Normally, 0.1 mg/kg to 25 mg/kg of at least one propofol derivative of the present invention is administered, but when applied locally, e.g. intracoronary administration, much lower total doses are also possible.

For practicing the methods of the present invention, said compound of the present invention is preferably administered by all possible routes (intraperitoneal, transdermal, intravenous, intravascular, intramuscular, inhalation), preferred route being as sterile solution for intravenous injection.

Thus, the esters of propofol according to the invention are useful as a medicament. Preferably, said compounds are used for the preparation of a medicament for anesthetizing a mammal or for treating and/or preventing convulsions, for treating and/or preventing migraine or related diseases or for inhibiting free radicals in a mammal.

A further aspect of the present invention relates to a pharmaceutical composition comprising at least one of the propofol derivatives according to the invention and a pharmaceutically acceptable carrier, more preferably comprising an α-proline propofol ester.

Another aspect of the invention relates to a kit comprising at least one of the propofol derivatives according to the invention in a dehydrated form, preferably in lyophilized form, and at least one physiologically acceptable aqueous solvent.

### FIGURES

FIG. 1 shows the structure of propofol (1) and propofol amino acid esters of the prior art (2a-c). A schematic diagram of the preferred method for preparing the compounds of the present invention is depicted in the middle of FIG. 1. The abbreviations used for reagents and substituents are well known to those in the art and explained in example 1. For further details, see example 1. Compounds 6a-d in combination with the substituents a-d at the end of FIG. 1 relate to preferred embodiments of the present invention.

FIG. 2 The plot in FIG. 2 shows an S (slope) versus log k′_{w} plot of the data obtained in the lipophilicity studies in example 3 and demonstrates that, equal to the polycratic capacity factors, slope values of the H-acceptors 2a-c are smaller than those of the amphiprotics 6a-d, proving the ability of the S parameter for encoding the total HB capacity of the compounds.

FIG. 3 shows the effects of propofol 1 and compounds 6a-d on [³⁵S]TBPS binding to unwashed rat cortical membranes. Rat cortical membranes were incubated with 2 nM [³⁵S]TBPS for 90 min in the presence of different concentrations of propofol 1, or compounds 6a, 6b, 6c, and 6d. The data is expressed as a percentage of binding measured in the presence of solvent and are means of two experiments.

FIG. 4 shows the modulatory action of compound 6a (a) and 6d (b) at human α1β2γ2 GABA_{A} receptors expressed in Xenopus laevis oocytes. Values are expressed as mean (6-13 different oocytes)±s.e.m. percentage of the potentiation of the control response to GABA (EC₂₀, 2-10 μM).

FIG. 5 shows the synthesis of activated precursors for use in subsequent synthesis of saccharide-conjugates with propofol.

FIG. 6 shows the synthesis of saccharide-conjugates with propofol either by direct conjugation (FIG. 6A) or via linker groups (FIG. 6B-D).

The following examples further illustrate the best mode contemplated by the inventors for carrying out their invention. The examples relate to preferred embodiments and are not to be construed to be limiting on the scope of the invention.

### EXAMPLES

Chemicals

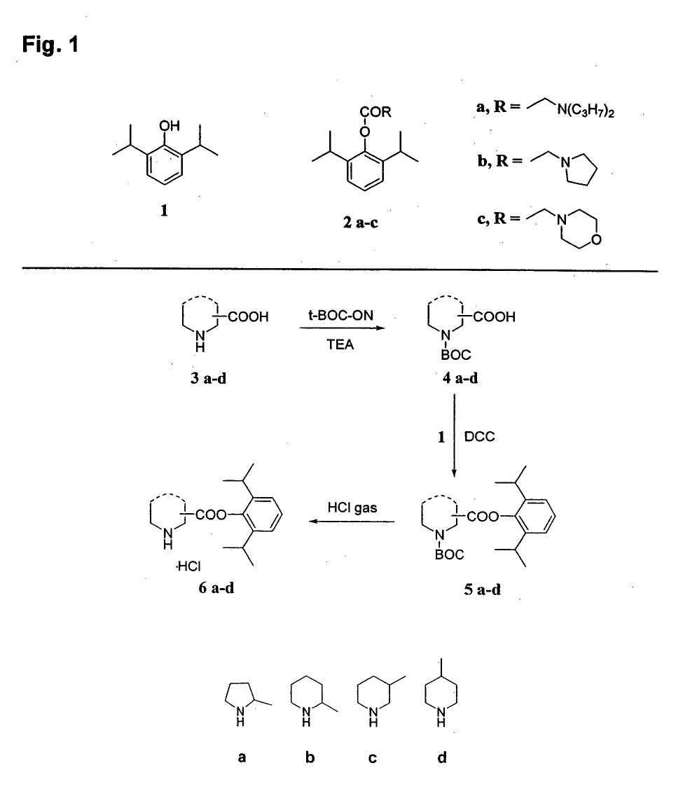

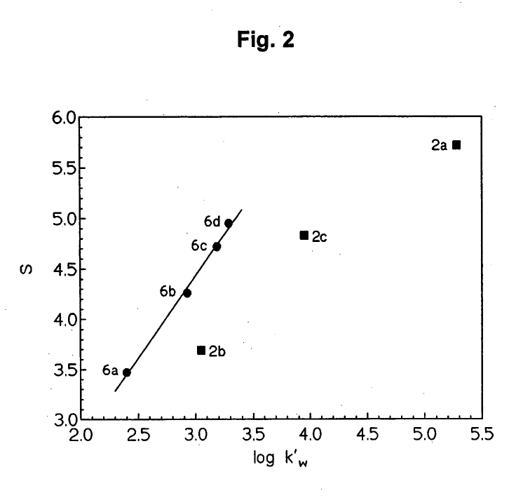

Propofol (1, see FIG. 1), dicyclohexylcarbodiimide (DCC), (S)-proline (3a, see FIG. 1), pipecolinic acid (3b, see FIG. 1), nipecotic acid (3c, see FIG. 1), isonipecotic acid (see FIG. 1 3d), and all other reagents were purchased from Sigma-Aldrich (Taufkirchen, Germany). Rat serum (lyophilized powder) and porcine liver esterase (suspension in 3.2 M (NH₄)₂SO₄ solution, pH 8) were also purchased from Sigma-Aldrich. Reagents used for the preparation of the buffers were of analytical grade. Fresh deionized water was used in the preparation of all the solutions.

Apparatus

Melting points were determined by the capillary method on a Buchi apparatus and are uncorrected. IR spectra were recorded as Nujol films for liquids and KBr pellets for solids on a Perkin-Elmer 283 spectrophotometer. ¹H-NMR spectra were recorded on a Varlan EM 390 spectrometer operating at 90 MHz (Varian, Milan Italy). Chemical shifts are expressed in δ values downfield from tetramethylsilane (TMS) used as internal standard. Mass spectra were recorded on a Hewlett-Packard 5995c GC-MS low resolution spectrometer (Hewlett-Packard, Milan, Italy) operating in electron impact mode. Elemental analyses were performed on a Hewlett-Packard 185 C, H, N analyzer and agreed with theoretical values to within ±0.40%. High-performance liquid chromatography (HPLC) analyses were carried out on a Waters Associates Model 600 pump equipped with a Waters 990 variable wavelength UV detector and a 20 μL loop injection valve (Waters, Milan, Italy). HPLC mobile phase was prepared using HPLC-grade methanol. For analysis, a Phenomenex C₁₈ column (25 cm×3.9 mm; 5 μm particles) was used as the stationary phase. A flow rate of 1 mL/min was maintained and the column effluent was monitored continuously at 210 or 270 nm. Quantification of the compounds was carried out by measuring the peak areas in relation to those of external standards. Stability studies were carried out at controlled temperature of 37±0.2° C. in a water bath.

Animals

Male Sprague-Dawley CD® rats (Charles River, Como, Italy) weighing 180-200 g were used. The animals were kept on a controlled light-dark cycle (light period between 8:00 a.m. and 8:00 p.m.) in a room with constant temperature (22±2° C.) and humidity (65%). Upon arrival at the animal facilities there was a minimum of 7 days of acclimation during which the animals had free access to food and water.

Animal care and handling throughout the experimental procedure were performed in accordance with the European Communities Council Directive of 24 Nov. 1986 (86/609/EEC). The experimental protocol were approved by the Animal Ethical Committee of the University of Cagliari.

### Example 1

### Synthesis of Cyclic Aminoacid Esters of Propofol

The propofol esters 6a-d were prepared according to the procedure illustrated in FIG. 1, by reacting the BOC-protected cyclic amino acids 4a-d with propofol 1 in the presence of DCC to give the corresponding esters 5a-d, which when deprotected with HCl gas yielded derivatives 6a-d as hydrochlorides (physical and spectral data of newly synthesized compounds 4a, 5a-d, and 6a-d are shown below in Table I).

BOC-protected amino acids: preparation of 1-(tert-butoxycarbonyl)proline (4a)

To a stirred mixture of proline (4.60 g, 40 mmol) in H₂O (25 mL) containing triethylamine (8.3 mL, 60 mmol), a solution of 2-(tert-butoxycarbonyloxyimino)-2-phenylacetonitrile (BOC-ON, 10.58 g, 43 mmol) in acetone (25 mL) was added. Stirring was prolonged for 12 h, and then 125 mL of a mixture of ethyl acetate:water (1:1, v/v) was added. The aqueous phase, combined with water (55 mL), used for washing the organic phase, was further washed with 50 mL of ethyl acetate, and then acidified with cold 0.1 N HCl (pH 2) to give compound 4a as a white precipitate.

N-BOC-piperidin carboxylic acids 4b-d were prepared in 87-89% yields, according to the above procedure (analytical data in agreement with those reported in literature (Ho B, Venkatarangan P M, Cruse S F, Hinko C. N, Andersen P H, Crider A M, Adloo M, Roane D S, Stables J P. 1998. Synthesis of 2-piperidinecarboxylic acid derivatives as potential anticonvulsants. Eur. J. Med. Chem. 33: 23-31. Bonina F P, Arenare L, Palagiano F, Saija A, Nava F, Trombetta D, De Caprariis P. 1998. Synthesis, stability, and pharmacological evaluation of nipecotic acid prodrugs. J. Pharm. Sci. 8: 561-567. Freund R. Mederski W K R. 2000. A convenient synthetic route to spiro[indole-3,4′-piperidin]-2-ones. Helv. Chim. Acta. 83: 1247-1255.).

Esterification of BOC-Drotected cyclic amino acids: preparation of 2.6-diisopropylphenyl 1-(tert-butoxycarbonyl)pirrolidin-2-carboxylate (5a) as a typical procedure

To a stirred solution of 1 (0.40 g, 2.25 mmol), compound 4a (0.57 g, 2.50 mmol), and dimethylaminopyridine (0.1 g, 0.82 mmol) in dry dichloromethane (15 mL), a solution of DCC (1.4 g, 6.8 mmol) in dry dichloromethane (10 mL) was added dropwise during 10 min. Stirring was continued at room temperature for 24 h, and then the dicyclohexylurea (DCU) precipitate was filtered off. The solution was evaporated under reduced pressure to give a residue which was purified by column chromatography on silica gel (petroleum ether-ethyl acetate 98:2 v/v as eluent) to give compound 5a.

Removal of the tert-butoxycarbonyl group: preparation of 2.6-diisopropylphenyl pirrolidin-2-carboxylate hydrochloride (6a) as a typical procedure

To a stirred and ice-cooled solution of ester 5a (0.50 g, 1.33 mmol) in chloroform (20 mL) HCl gas was bubbled for 5 min. Evaporation of the solvent under reduced pressure gave compound 6a as a white solid. t,0220

### Example 2

### Solubility of Cyclic Aminoacid Esters of Propofol

The solubility of the propofol derivatives 6a-d (6b-d as hydrochloride salts) in deionized water at 25° C. was determined by adding excess amount of compound to 1-2 mL of water in screw-capped test tube. The resulting mixture was vortexed for 10 min and then mechanically shaken in a thermostatic bath shaker (100 rpm) for 72 h to attain equilibrium. Next, the mixture was filtered through a 0.45 μm membrane filter (Millipore®, cellulose acetate) and an aliquot was diluted with an appropriate amount of water and analyzed for the aminoacid ester prodrug content spectrophotometrically at 210 nm. All of the manipulations were made without removal of the test tubes from the water bath, using thermostated pipettes, syringes, and buffer solutions. In Table II, as shown below, solubility data is compared with the data previously determined for propofol derivatives 2a-c (Trapani G. Latrofa A, Franco M, Lopedota A, Maciocco E, Liso G. 1998. Water-soluble salts of amino acid esters of the anesthetic agent propofol. Int. J. Pharm. 175: 195-204.).

TABLE II
Aqueous solubility, , stability in physiological
media, and GABA_{A} receptor binding of amino acid
esters (as hydrochloride salts) of propofol

| | Solubility | Half-lives at 37° C.^{a} | | | | | |
|---|---|---|---|---|---|---|---|
| | (mg/mL) | | | | Porcine liver | | [³⁵S]TBPS |
| | in deionized | pH 7.4 | 50% rat | | esterase | | binding |
| Cmp | water^{a} | buffer | serum | | (13 U/mL) | | IC₅₀, μM^{e,g} |
| 6a | 350.0 | 6 h | 17 | min | 17 | min | 31.7 |
| 6b | 13.4 | 7 h | 2.5 | min | 13 | min | 39.5 |
| 6c | 525.0 | ^{f} | 6 | h | 3 | h | 152 |

| | | | | | | |
|---|---|---|---|---|---|---|
| 6d | 29.7 | ^{f} | ^{b,f} | 45 | h | ^{h} |
| 2a | <0.058^{b,c} | ^{b,f} | | | | ^{b,i} |
| 2b | 0.735^{b} | ^{b,f} | ^{b,f} | | | 6.52^{a} |
| 2c | 0.213^{b} | ^{b,f} | ^{b,f} | | | ^{b,i} |

^{a}Data are means of two determination (less than 10% of difference).
^{b}Previously determined in phosphate buffer at pH 7.4.
^{c}Determined as free base.
^{f}Stable after 48 h.
^{g}[³⁵S]TBPS binding in unwashed rat brain; for propofol IC₅₀ = 4.17 μM.
^{h}No displacement, but a % increase in [³⁵S]TBPS binding was observed.
ⁱNo displacement.

Compared to propofol, whose solubility under the same conditions was about 0.15 mg/mL, two derivatives, prolinate 6a and nipecotate 6c, their solubilities being 350 and 525 mg/mL, respectively, afforded a strong increase of the aqueous solubility of the anesthetic drug. None of the equations proposed for computing intrinsic solubilities gave accurate predictions (Peterson D L, Yalkowsky S H. 2001. Comparison of two methods for predicting aqueous solubility. J. Chem. Inf. Comput. Sci. 41: 1531-1534. Teitko I V, Yu V, Kasheva T N, Villa A E P. 2001. Estimation of aqueous solubility of chemical compounds using E-state indices. J. Chem. Inf. Comput. Sci. 41: 1488-1493.), though it was apparent that the most relevant differences in solubility of derivatives 6 could be, at least in part, accounted for by large differences in crystal lattice energies. In fact, the most soluble 6a and 6c have a melting point lower than 200° C. whereas pipecolinate 6b and isonipecotate 6d melt at 225 and 234° C., respectively.

### Example 4

### Chemical Hydrolysis of Cyclic Aminoacid Esters of Propofol

The hydrolysis of the propofol esters 6a-d was studied in aqueous buffer solutions (0.05 M phosphate buffers; ionic strength of 0.5 maintained by adding a calculated amount of KCl) at pH values of 4, 6, and 7.4 and temperature of 37±0.2° C. The reactions were initiated by adding 100 μl of a stock solution of the ester (13 mg/mL methanol) to 20 mL of the buffer solution preheated at 37° C., in screw-capped test tubes (final concentration about 2.0×10⁻⁴ M). The solutions were kept in a water bath at a constant temperature, and at appropriate intervals aliquots of 20 μL were withdrawn and analyzed by HPLC. Pseudo-first-order rate constants for the hydrolysis were determined from the slopes of linear plots of the logarithm of residual propofol ester against time.

### Example 5

### Hydrolysis of Cyclic Aminoacid Esters of Propofol in Physiological Solution

The susceptibility of the derivatives 6a-d to undergo conversion to the parent propofol was studied in 0.05 M phosphate buffer (pH 7.4) containing 50% of rat serum at 37° C. Each reaction were initiated by adding 100 μL of the methanolic stock solution of compound under examination to 1.6 mL of preheated serum solution (final concentration about 1×10⁻³ M) and the mixture was maintained in water bath at 37° C. At appropriate times, 100 μL samples were withdrawn and added to 500 μL of cold acetonitrile in order to deproteinize the serum. After mixing and centrifugation (10 min at 4000 rpm), 20 μL of the clear supematant were filtered through 0.2 μm membrane filter (Waters, PTFE 0.2 μm) and analyzed by HPLC.

Hydrolysis of compounds 6a-d in the presence of porcine liver esterase was followed using a reported procedure (Bonina F P, Arenare L, Palagiano F, Saija A, Nava F, Trombetta D, De Caprariis P. 1998. Synthesis, stability, and pharmacological evaluation of nipecotic acid prodrugs. J. Pharm. Sci. 8: 561-567.).

Results:

The kinetics of hydrolysis of the derivatives 6a-d were determined in 0.05 M phosphate buffers at pHs 4.0, 6.0 and 7.4 at 37° C. as well as in rat serum solution and in the presence of porcine liver esterase.

All the examined derivatives were stable at pH values of 4.0 and 6.0 for 48 h, whereas at physiological pH the hydrolysis of prolinate 6a and pipecolinate 6b followed first-order kinetics with half-lives of 6 and 7 h, respectively. The derivatives 6a and 6b, but not 6c and 6d, were found to be cleaved quantitatively to the parent drug in rat serum and porcine liver esterase solutions at 37° C., and the observed half-lives are reported in Table II. Kinetic data showed that 6a and 6b are stable enough in solution buffered at pH 7.4, their half-lives exceeding 6 h, but undergo a fast cleavage at conditions similar to those prevailing in vivo, providing propofol within few minutes. Conversely, compounds 6c and 6d were found to be stable enough both in buffer solution and less susceptible than the α-amino acid esters to esterases' catalysis. The observed high stability toward the chemical hydrolysis can be ascribed to the steric protection of the C(O)O— bond by bulky flanking diisopropyl groups on the phenyl ring. The fact that the proline (6a) and pipecolinic acid (6b) esters, similarly to α-amino acid esters or related short-chained aliphatic amino acid esters (Bundgaard H, Larsen C, Thorbek P. 1984. Prodrugs as drug delivery systems. XXVI. Preparation and enzymic hydrolysis of various water-soluble amino acid esters of metronidazole. Int. J. Pharm. 18: 67-77.), are less resistant than compounds 6c and 6d to chemical and enzyme-catalyzed hydrolysis could result from either the electron withdrawing effect of the protonated amino group, which activates the ester linkage toward OH⁻ attack, and (predominantly) the intramolecular catalysis (i.e., intramolecular N→CO 1, 2 proton shift) by the neighboring amino group (protonated or not protonated) that promotes ester cleavage. The above finding demonstrates that prolinate 6a is highly soluble, stable in water at physiological pH and rapidly hydrolyzed in plasma. Therefore, compound 6a is an excellent prodrug of propofol for parenteral administration.

### Example 6

### In vitro [³⁵S]TBPS Binding Assay

Experimental set up:

Rats were killed by decapitation and their brains rapidly removed on ice. The cerebral cortex was dissected out and homogenized in 50 volumes of ice-cold 50 mM Tris-citrate buffer (pH 7.4 at 25° C.) containing 100 mM CaCl₂ using a Polytron PT 10 (setting 5, for 20 sec) and centrifuged at 20.000×g for 20 min. The resulting pellet was resuspended in 50 volumes of 50 mM Tris-citrate buffer (pH 7.4 at 25° C.) and used for the assay. [³⁵S]TBPS binding was determined in a final volume of 500 μL consisting of. 200 μL of tissue homogenate (0.20-0.25 mg protein), 50 μL of [³⁵S]TBPS (final assay concentration of 1 nM), 50 μl 2 M NaCl, 50 μL of drugs or solvent and buffer to volume. Incubations (25° C.) were initiated by addition of tissue and terminated 90 min later by a rapid filtration through glass-fiber filter strips (Whatman GF/B, Clifton, N.J.), which were rinsed twice with a 4 mL portion of ice-cold Tris-citrate buffer using a Cell Harvester filtration manifold (model M-24m Brandel, Gaithersburg, Md.). Filter bound radioactivity was quantitated by liquid scintillation spectrometry. Nonspecific binding was defined as binding in the presence of 100 μM picrotoxin and represented about 10% of total binding. Protein content was determined by the method of Lowry²⁰ using bovine serum albumin as a standard.

Results:

Receptor binding.

GABA_{A} receptors are sensitive targets for the action of propofol and other general anesthetics (Trapani G, Altomare C, Sanna E, Biggio G, Liso G. 2000. Propofol in anesthesia. Mechanism of action, structure-activity relationships, and drug delivery. Curr. Med. Chem. 7: 249-271. Franks N P, Lieb W R. 1994. Molecular and cellular mechanisms of general anaesthesia. Nature (Lond). 367: 607-614.). Binding of [³⁵S]TBPS, a cage convulsant which binds in close proximity to the chloride channel portion of the GABA_{A} receptor at level of the picrotoxin binding site, constitutes a tool for studying the function of the GABA_{A} receptor complex (Squires R F, Casida J E, Richardson M, Saederup E. 1983. [³⁵S]t-Butylbicyclophosphorothionate binds with high affinity to brain-specific sites coupled to γ-aminobutyric acid-A and ion recognition sites. Mol. Pharmacol. 23: 326-336). Propofol, mimicking the action of other general anesthetics, such as alphaxalone and pentobarbital (Concas A, Santoro G, Serra M, Sanna E, Biggio G. 1991. Neurochemical action of the general anaethetic propofol on the chloride ion channel coupled with GABA_{A} receptor. Brain Res. 542: 225-232.), reduces [³⁵S]TBPS binding in a concentration-dependent manner.

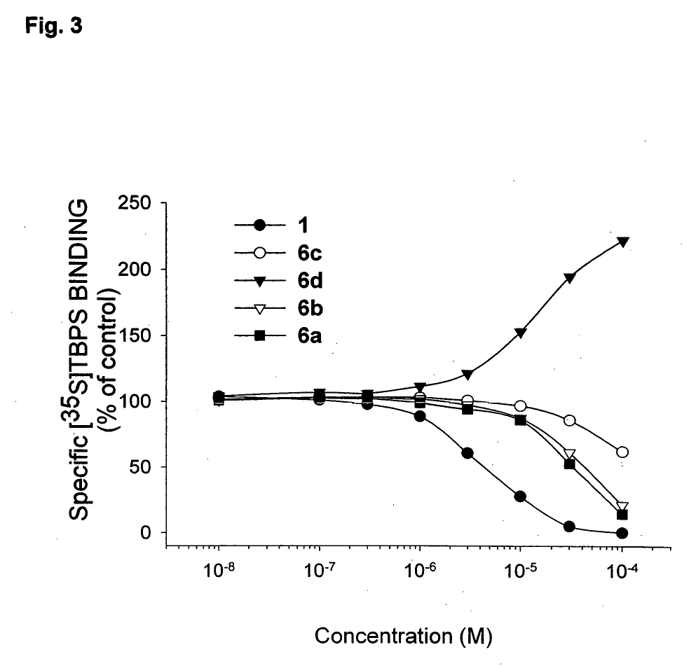

The ability of the compounds 6a-d to interact with [³⁵S]TBPS binding sites was measured and compared with that of propofol. Affinity data, expressed as IC₅₀ values (see Table II above), demonstrates that compounds 6a and 6b are able to reduce the [³⁵S]TBPS binding, with IC₅₀ values one magnitude order higher than IC₅₀ value of propofol (4.17 μM). A similar effect, at doses higher than 100 μM, was shown by nipecotate 6c, whereas compound 6d displayed an increase of [³⁵S]TBPS binding, an effect similar to that of the antagonist bicuculline (Concas A, Sanna E, Mascia M P, Serra M, Biggio G. 1990. Diazepam enhances bicuculline-induced increase of t-[³⁵S]butylbicyclophosphorothionate binding in unwashed membrane preparations from rat cerebral cortex. Neurosci. Lett. 112: 87-91.). FIG. 3 shows the competitive inhibition curves of the eXarnined cyclic amino acid ester derivatives.

### Example 7

### Electrophysiological Measurements Using Xenopus Oocytes

Experimental set up:

Complementary DNAs encoding the human α1, β2, and γ2 GABA_{A} receptor subunits were subcloned into the pCDM8 expression vector (Invitrogen, San Diego, Calif.). The cDNAs were purified with the Promega Wizard Plus Miniprep DNA Purification System (Madison, Wis.) and then resuspended in sterile distilled water, divided into portions, and stored at −20° C. until used for injection. Stage V and VI oocytes were manually isolated from sections of Xenopus laevis ovary, placed in modified Barth's saline (MBS) containing 88 mM NaCl, 1 mM KCl, 10 mM Hepes-NaOH buffer (pH 7.5), 0.82 mM MgSO₄, 2.4 mM NaHCO₃, 0.91 mM CaCl₂, and 0.33 mM Ca(NO₃)₂ and treated with 0.5 mg/mL of collagenase Type IA (Sigma) in collagenase buffer (83 mM NaCl, 2 mM KCl, 1 mM MgCl₂, 5 mM Hepes-NaOH buffer, pH 7.5) for 10 min at room temperature, to remove the follicular layer. A mixture of GABA_{A} receptor α1, β2, and γ2 subunit cDNAs (1.5 ng/30 nL) was injected into the oocyte nucleus using a 10 μL glass micropipette (10-15 μm tip diameter). The injected oocytes were cultured at 19° C. in sterile MBS supplemented with streptomycin (10 μg/mL), penicillin (10 U/mL), gentamicin (50 μg/mL), 0.5 mM theophylline, and 2 mM sodium pyruvate. Electrophysiological recordings began approximately 24 h following cDNA injection. Oocytes were placed in a 100-μL rectangular chamber and continuously perfused with MBS solution at a flow rate of 2 mL/min at room temperature. The animal pole of oocytes was impaled with two glass electrodes (0.5 to 3 MΩ) filled with filtered 3 M KCl and the voltage was clamped at −70 mV with an Axoclamp 2-B amplifier (Axon Instruments, Burlingame, Calif.). Currents were continuously recorded on a strip-chart recorder. Resting membrane potential usually ranged from −30 to −50 mV. Drugs were perfused for 20 s (7-10 s were required to reach equilibrium in the recording chamber). Intervals of 5 to 10 min were allowed between drug applications.

Results:

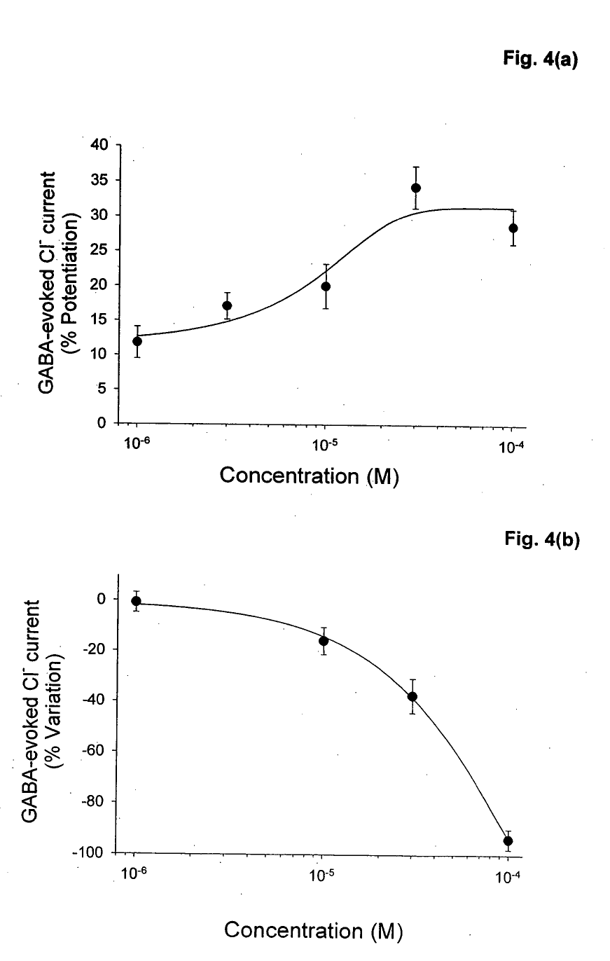

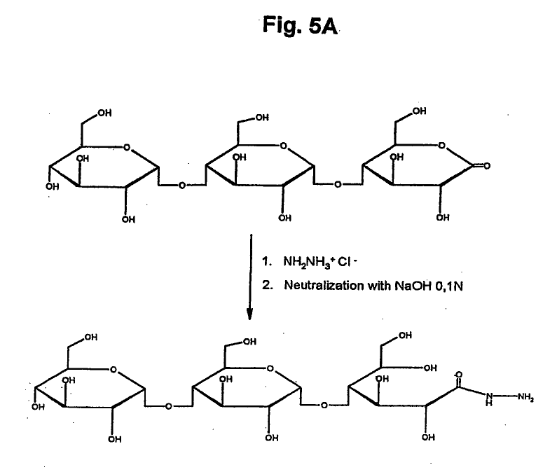

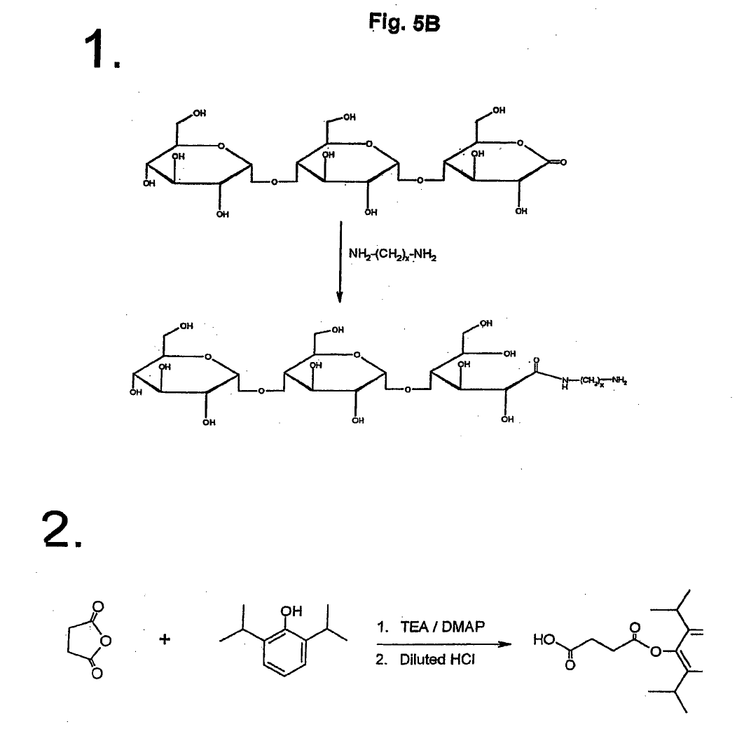

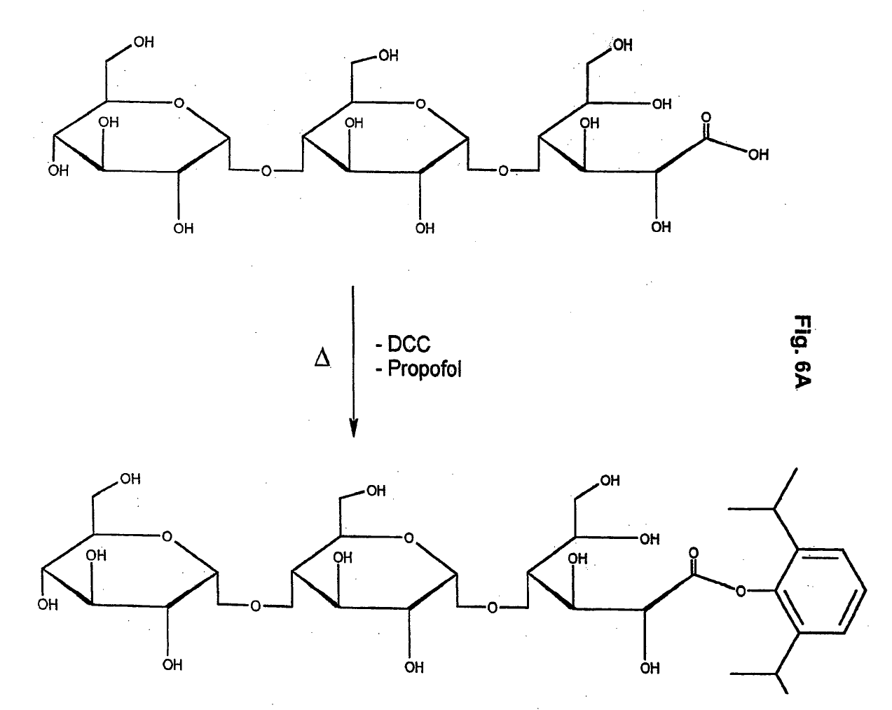

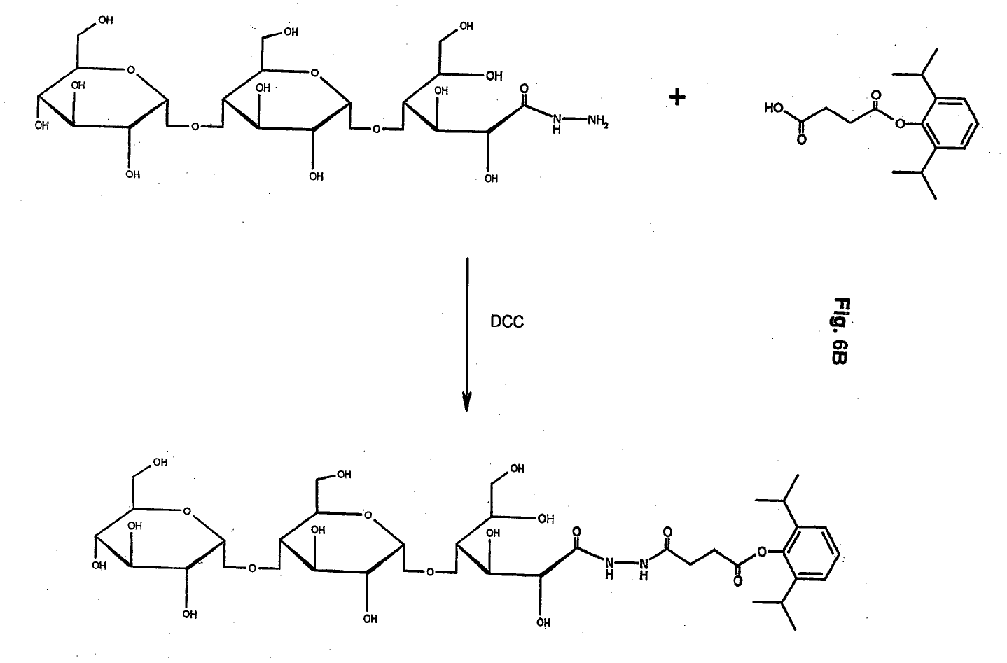

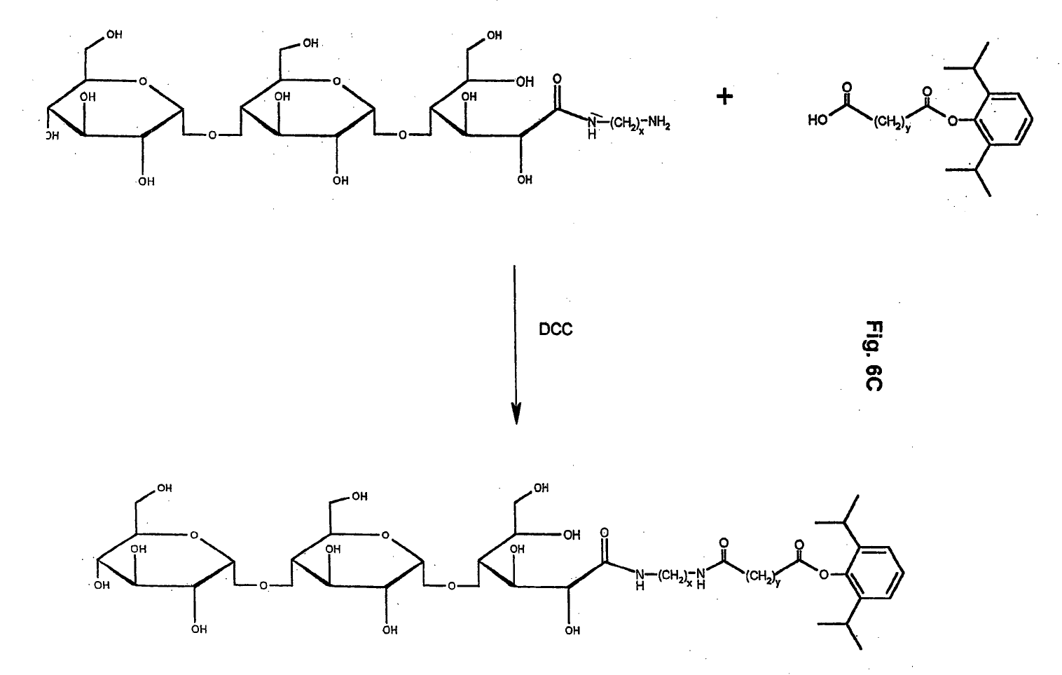

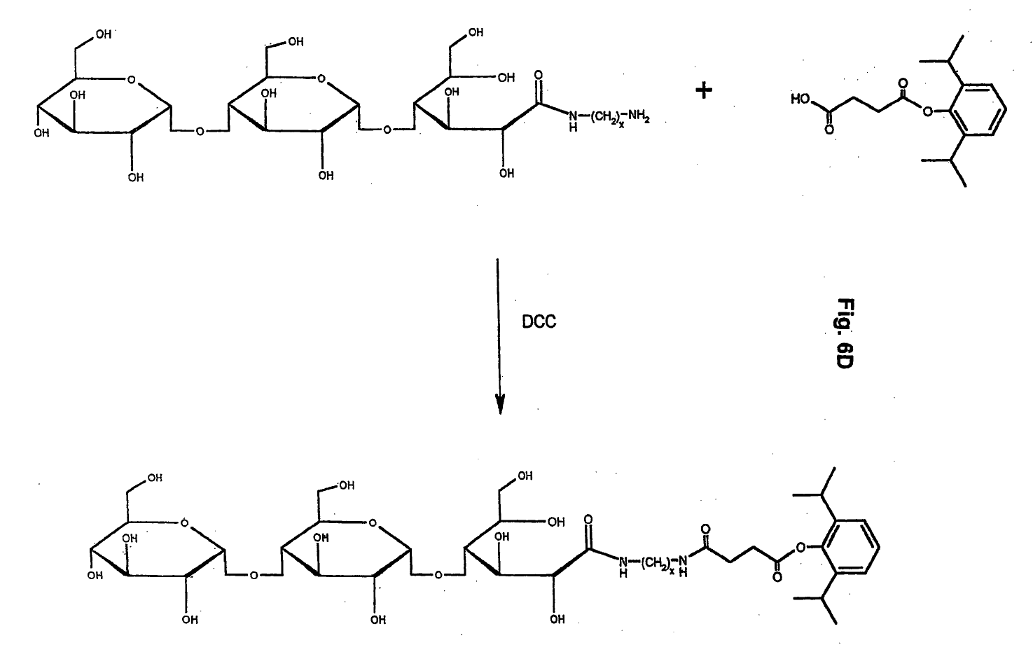

Expression of human α1, β2, and γ2 GABA_{A} receptor subunit constructs in Xenopus-laevis oocytes was utilized in a voltage-clamp electrophysiological assay. FIG. 4 shows the profiles of prolinate 6a and isonipecotate 6d. Consistent with binding data, GABA-evoked chloride currents elicited at cloned GABA_{A} receptors were enhanced by 6a and diminished by 6d, both in a concentration-dependent manner with their maximal effects apparent at the concentration of 50 and 100 μM, respectively.

Taken together, the in vitro results demonstrated that all the ester derivatives 6a-d modulate GABA_{A} receptors and possess intrinsic activity, though lower than that of the parent compound 1. Three amino acid esters, 6a-c, behaved like propofol, whereas isonipecotic acid ester revealed a bicuculline-like profile.

### Example 8

### In vivo Screening of Anticonvulsant and Anesthetic Activities

Experimental set up:

Rats received an intraperitoneal administration of propofol 1 (40 mg/kg, suspended in saline with a drop of Tween 80 per 5 mL) and equimolar doses of compounds 6a and 6d. The anticonvulsant activity against pentylenetetrazole-induced seizures (55 mg/kg) was measured. Rats treated with pentylenetetrazole were considered “protected” when clonic or tonic seizures and death did not occur.

The loss and reestablishment of righting responses, time of anesthesia induction, and sleeping time were also assessed. Rats (five per group) were treated with propofol and Diprivan®, both at a dose of 60 mg/kg, and an equimolar dose of compound 6a (105 mg/kg) and were continuously monitored for the loss of righting reflex (onset and duration). Propofol and its derivative 6a were dissolved in saline with a drop of Tween 80 per 5 mL and administered intraperitoneally in a volume of 0.3 mL per 100 g of body mass. Anesthesia induction (sleep onset) was defined as the time from drug administration to loss of righting reflex, whereas the sleeping time was the time from the loss of the righting reflex until the animals were plantigrade on all four legs. The significance of differences in behavioral data were analyzed by the ANOVA test.

Results:

Compounds 6a and 6d, displaying in vitro agonist and antagonist behavior, respectively, were tested in vivo for their anticonvulsant activity, whereas 6a, the derivative showing the best prodrug properties, was compared to propofol, administered either as an oil/water emulsion or as the commercial formulation Diprivan®, for the in vivo anesthetic activity. As shown in Table III, compound 6a, like propofol, protected completely the animals from the pentylenetetrazole-induced convulsions. In contrast with its in vitro GABA_{A} antagonist behavior (see FIGS. 3 and 4), compound 6d appeared to protect animals, though only 60%, against convulsions. Among the hypotheses that may be formulated to explain this result, it may not be excluded that propofol isonipecotate 6d, stable in vitro in rat serum solution, can instead be hydrolyzed in vivo, releasing propofol and isonipecotic acid (Anderson A, Belleli D, Bennett D J, Buchanan K J, Casula A, Cooke A, Feilden H, Gemmel D K, Hamilton N M, Hutchinson E J, Lambert J J, Maldment M S, McGuire R, McPhall P, Miller S, Muntoni A, Peters J A, Sansbury F H, Stevenson D, Sundaram H. 2001. α-Amino acid phenolic esters derivatives: novel water-soluble general anesthetic agents which allostercally modulate GABA_{A} receptors. J. Med Chem. 41: 3582-3591.),¹⁵ a known GABA_{A} agonist, at anticonvulsant concentrations.

The anesthetic activity of compound 6a was investigated by measuring onset and duration of loss of righting reflex, in comparison with that elicited by the clinical propofol formulation (Diprivan®), and an oil/water emulsion of 1 in the presence of Tween 80 (Table III). Induction time of loss of righting reflex subsequent to intraperitoneal administration of compound I was notably shorter than that observed for Diprivan®. Compound 6a showed an induction time intermediate between the emulsion formulation and Diprivan®, whereas the duration of anesthesia followed the order propofol emulsion <6a≈Diprivan®. Therefore, compound 6a could be considered an efficacious anesthetic with the same duration of action of Diprivan® but a considerably shorter induction time than the marketed formulation.

TABLE III
In vivo anticonvulsant and anesthetic activities
of propofol ester derivatives

| | Anticonvulsant activity^{a} | Loss of righting | |
|---|---|---|---|
| | No. of rats | reflex, LRR (sec)^{b} | |
| Compounds | protected/tested | Onset | Duration |
| 1 | 10/10 | 114.4 ± 9.5 | 2245 ± 252 |
| 6a | 10/10 | 162.7 ± 4.3^{c,d} | 2403 ± 592 |
| 6d | 6/10 | | |
| Diprivan ® | | 289 ± 14.8^{c} | 3895 ± 1113 |

^{a}Protection against clonic and tonic seizures induced by pentylenetetrazole (55 mg/kg, i.p.). Compounds 6a and 6d were tested at doses equimolar to 40 mg/kg propofol.
^{b}Anesthetic activity measured as onset and duration of LRR (mean ± s.e.m.). Compound 6a was administered i.p. at a dose of 105 mg/kg equimolar to 60 mg/kg dose of propofol;
^{c}p < 0.01 vs. propofol-treated animals,
^{d}p < 0.01 vs. Diprivan-treated animals.

### Example 9

### Synthesis of Saccharide-Conjugates of Propofol

In a 50 ml round bottom flask 1 ml of propofol was mixed with 2.5 ml TEA at room temperature. When the mixture seemd homogeneous 5.5 mmol of succinnic anhydride were added. The reaction was allowed to proceed under moderate stirring conditions for 22 h. The reaction was followed by TLC monitoring or simply observing the disappearance of succinnic anhydride whose solubility in the mixture is low, so most of it remained in the reaction vessel as a white solid. After 22 h the reaction was stopped and the solution looked brownish. After elimination of most of the TEA under vacuum, 10 ml of 0.2N HCl were added to the solution which was vigorously stirred and kept in an ice bath for 30 min. Thereafter, a white swaying precipitate was removed from the reaction by filtration on a proper funnel filter. The precipitate was dissolved once more in EtOH and was precipitated a second time by adding cold water, filtrated and kept at −20° C.

Three grams of lactobionic acid were dissolved in 5 ml of warm DMSO (−70° C.). After the complete dissolution 7.5 mmol of mono chloride salt of hydrazine were added to the reaction vessel. The solution was stirred at 45° C. for 20 h. The proceeding of the hydrazide formation was monitored using TLC coupled with a ninhydrin test to reveal the presence of amino groups. The protonated amine turned yellow in the ninhydrine test. When the reaction was complete, an excess of water and 0.1 N NaOH was added dropwise until a pH˜10 was reached. The mixture was frozen and lyophilised. The dry product may be dissolved in water and lyophilised once more to eliminate the last traces of DMSO.

Alternatively, the reaction mixture can be diluted with water, lyophilised and then be incubated overnight on AgCO₃ to eliminate the chloride ions. Before making the last lyophilisation a short passage through cation exchanger resins may be run to get rid of possible Ag⁺ ions.

One mmol of the succinnic acid mono-propofol ester and 370 mg of the lactobionic acid hydrazide were dissolved in 3 ml of DMF and stirred at room temperature. A 1:1 molar amount of DCC was added to the solution and the temperature was decreased to 0° C. The reaction was allowed to run one hour under these conditions before switching gradually the temperature to 25° C. The reaction was monitored by TLC coupled with a ninhydrin test. The disappearing of the amino functions indicated the end of the reaction (normally after 2 h). The reaction was then stopped by adding dilute HCl. The precipitate was washed three times with cold water and then eliminated. The aqueous fractions were frozen and lyophilised. The purity of the product was checked by TLC.

Propofol—Maltotriose Prodrug

In 10 ml of a 3:1 DMSO:MeOH mixture 200 mg of Propofol were dissolved as well as a three times molar excess of maltotrionic acid, and a catalytic amount of DMAP (dimethylamino pyridine). The solution was left stirring at room temperature for 10 min. In a separate vessel 350 mg of DCC were dissolved in 5 ml of the same solution and added to the previous mixture dropwise in a time period of 10 min. The reaction was allowed to run under the same conditions for 20 h and was then stopped and filtrated. The coupling product was recovered by precipitation in acetone (50 ml) and washed several times with EtOH (100 ml), AcOEt (100 ml) and finally acetone (100 ml). The reaction was monitored by TLC and the purity of the product was also confirmed by RP-HPLC on a C-18 column.

Propofol—oxHES10 kD Prodrug

In 10 ml of a 5:1 DMSO:MeOH mixture 200 mg of Propofol were dissolved as well as a three times molar excess of oxHES10 kD, and a catalytic amount of dimethylamino pyridine. The solution was left stirring at the temperature of 40° C. In a separate vessel 350 mg of DCC were dissolved in 5 ml of the same solution and added to the previous mixture dropwise in a time period of 10 min. The reaction was allowed to run under the same conditions for 30 h and then stopped and filtrated. The coupling product was recovered by precipitation in acetone (50 ml) and washed several times with MeOH (100 ml), AcOEt (100 ml) and finally acetone (100 ml). The reaction was monitored by TLC and the purity of the product was confirmed by RP-HPLC on a C-18 column.

### Example 10

### Selective Oxidation of Maltotriose Reducing End

In a round bottom flask one gram of maltotriose (˜2 mmol) was dissolved in distilled water (1.0 ml). Thereafter 2.0 ml of a 0.1 N I₂ solution were added and the solution became brown. A 2 ml pipette containing 2.0 ml 1 N NaOH solution was then connected to the flask using a two ways connector, and the NaOH solution was dropped in, once every four minutes (each drop having the volume of ˜20 μl). After adding almost 0.2 ml of the NaOH the solution started to become clear again, then the second 2 ml portion of 0.1 N I₂ solution had to be added. At the end of this process 50 ml a 0.1 N I₂ solution and 7.5 ml of 1 N NaOH solution was used.

The reaction was then stopped, acidified with 2.0 N HCl solution, and extracted several times with ethyl ether in order to remove any I₂ left. At the end the solution was passed directly through the cation exchanger IR-120 H⁺, and then incubated overnight in presence of silver carbonate in order to eliminate any excess of iodine/iodide. Thereafter the filtrate was passed once more through the same cation exchanger before being lyophilised. The final yield was found to be 85% and 95%.

At 70° C., for 24 h under argon atmosphere and moderate stirring. The reaction was stopped by adding cold acetone which precipitates the conjugate. After centrifugation the solid pellet is resuspended in acetone several times until the filtrate did not show any more red coloration. The pellet is finally dissolved in water and lyophilised. The purity of the coupling product was checked by RP-HPLC and the drug content was determined by UV photometry. The coupling product contains 0.4 μg Daunorubicin per mg. The chemical yield was 78%.

### Example 11

### Coupling of Propofol to Lactobionic Acid

a) Synthesis of Succinnic Acid Mono-Propofol Ester

In a 50 ml round bottom flask 1 ml of propofol has been stirred with 2.5 ml of TEA at room temperature. When the mixture looked homogeneous 5.5 mmol of succinnic anhydride were added. The reaction was allowed to proceed under moderate stirring conditions for 22 h. The progress of the reaction was followed by TLC monitoring or by simply observing the disappearance of succinnic anhydride whose solubility in the mixture is low, so most of it remained in the reaction vessel as a white solid. After 22 h the reaction was stopped, the solution looked brownish. After elimination of most of the TEA under vacuum, 10 ml of 0.2 N HCl were added to the solution which was vigorously stirred and kept in an ice bath for 30 min. Thereafter the white swaying precipitate was removed from the reaction by filtration through a proper funnel filter. The precipitate was dissolved once more in EtOH and precipitated a second time by adding cold water, filtrated and kept at −20° C.

b) Synthesis of Lactobionic Acid Hydrazide

Three grams of lactobionic acid were dissolved in 5 ml of warm DMSO (˜70° C.). After the complete dissolution 7.5 mmol of mono chloride salt of hydrazine were added to the reaction vessel. The solution was stirred at 45° C. for 20 h. The proceeding of the hydrazide formation was monitored using TLC coupled with a ninhydrin test to reveal the presence of free amino groups. The protonated amine appeared yellow in the ninhydrin test. When the reaction seemed complete, it was stopped by adding an excess of water and then 0.1 N NaOH solution was inserted dropwise until a pH˜10 was reached in order to neutralise the HCl. The mixture was frozen and lyophilised. The dry product was then dissolved in water and lyophilised once more to eliminate the last traces of DMSO.

c) Alterative Synthesis of Lactobionic Acid Hydrazide

Three grams of lactobionic acid lacton were dissolved in 5.0 ml of warm DMSO (˜70° C.). Once dissolved 1.0 gram of BOC-Hydrazine was added to the reaction vessel. The reaction ran for 16 h under inert atmosphere (argon) and was monitored by TLC (eluent CH₃Cl). When the spot of the BOC-hydrazine disappeared the reaction was stopped, cooled down to 4-5° C. and extracted with water—chloroform several times. The aqueous phase was finally degassed and lyophilised. The product dissolved in MeOH has been deprotected from the BOC-function by bubbling HCl gas into the solution for 30′. The deprotection was also monitored by TLC. The hydrochloride salt was characterised by ESI-MS.

### d) Synthesis of Lactobionic Acid Diamino Butanamide

Three grams of lactobionic acid lacton were dissolved in 3.0 ml of warm DMSO (˜70° C.). In a separate vessel a 30 times molar excess of diamino butane was dissolved in 2.0 ml of DMSO and then added to the first solution. The reaction was left under argon overnight under moderate stirring. The monitoring of the reaction was done by TLC. After stopping the reaction by adding 30 ml of NaOH sol. 0.01 N, this solution was extracted with a mixture chloroform/ethyl acetate 4:1 several times. The organic phase, washed two times with water was eliminated, while the aqueous phase, after degassing, was lyophilised. The product showed the calculated mol peak in ESI-MS.

e) Final Coupling

One mmol of the succinnic acid mono-propofol ester and one mmol of the lactobionic acid amino derivative (from reaction b, c, or d) were dissolved in 3 ml of DMF and stirred at room temperature. The temperature was decreased to 0° C. and a 1:1 molar amount of DCC was added to the chilled solution. The reaction was allowed to run one hour under these conditions before increasing gradually the temperature to 25° C. The reaction was monitored by TLC coupled with a ninhydrin test. The disappearing of the free amino functions indicated the end of the reaction (normally after 2 h). The reaction was then stopped by adding dilute HCl. The precipitate was washed three times with cold water and then eliminated. The aqueous fractions were frozen and lyophilised. The purity of the product was checked by TLC, confirmed by RP-HPLC (C₁₈), and the characterisation has been done by ESI-MS. Solubility of the product in water at 25 was greater than 800 mg/ml, reflecting more than 200 mg propofol content in this solution.

### Example 12

### Coupling of Propofol to Glucosamine

In a two necked round bottom flask 1.8 mmol of succinnic acid mono-propofol ester is dissolved in 2.0 ml of MeOH. The solution is then chilled in an ice bath. A 5 times molar excess of CDI is then added to the solution and allowed to run in the same conditions for 15 min. With the help of a dropping funnel an equimolar solution of glucosamine in 2 ml of a 3:1 mixture DMF:MeOH was slowly added during 10 min. Thereafter the reaction was allowed to proceed for one more hour on ice and then overnight at room temperature. The reaction is monitored by TLC. The reaction was finally stopped by adding 10 ml of a cold 0.1 N HCl solution, filtered and passed through a cation exchanger column filled with IR-120 H⁺. The eluate is finally lyophilised and the purity is checked by RP-HPLC. The product was characterised by ESI-MS and NMR.

### Example 13

### Coupling of Propofol to Maltotrionic Acid

In 2.0 ml of a 3:1 DMSO:MeOH mixture were dissolved 200 mg of Propofol, a three times molar excess of maltotrionic acid, and a catalytic amount of TEA. The solution was left stirring at room temperature for 10 min. In a separate vessel 350 mg of DCC were dissolved in 1 ml of the same solution and added dropwise to the previous mixture during a 3 min. time period. The reaction was warmed up to 60° C. and allowed to run under these conditions for 20 h. Finally it was stopped and then filtrated. The coupling product was recovered by precipitation in acetone (50 ml) and washed several times with EtOH (100 ml), AcOEt (100 ml) and-finally acetone (100 ml). The reaction has been monitored by TLC and the purity of the product has been confirmed also by RP-HPLC on a C-18 column.

### Example 14

### Coupling of Propofol to Glucuronic Acid

In a two necked 50 ml round bottom flask 10 mmol of glucuronic acid were dissolved in 2.0 ml of DMF. An equimolar amount of TEA was added and the solution was cooled down in an ice bath. Then 12 mmol of isobutyl chlorocarbonate were added and the reaction was kept cold for 30 min. In a separate vessel 10 mmol of propofol were mixed with with 0.5 ml of TEA and then added dropwise to the first solution with the help of a dropping funnel. The reaction run for 1 day at 4° C. and overnight at room temperature. It was monitored by TLC. After stopping the run the solution was evaporated yielding a brown oily product which was dissolved in water and extracted several times with chloroform. The organic phase, washed two times with water can be eliminated. The aqueous phase, after degassing, was passed through a mixed ion exchanger before being lyophilised. The purity was checked by RP-HPLC and the product has been characterised by ESI-MS and NMR.

1. A propofol derivative comprising the formula: 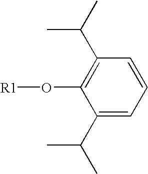
wherein R1 is a cyclic or linear amino acid or oligo amino acid, which may be fused to an aromatic or heterocyclic ring, and wherein the propofol derivative is present in the form of a free base or a salt.

2. The propofol derivative according to claim 1, wherein the amino acid is C-terminally linked to propofol.

3. The propofol derivative according to claim 1 comprising the formula 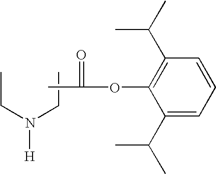
wherein the heterocyclic group comprises 4 to 5 methylene groups and wherein the heterocyclic group is optionally further substituted.

4. The propofol derivative according to claim 1, wherein R1 is selected from the group consisting of proline, pipecolinic acid, nipecotic acid and isonipecotic acid.

5. The propofol derivative according to claim 1, wherein R1 is selected from the group consisting of α-proline, α-pipecolinic acid, and β-nipecotic acid.

6. The propofol derivative according to claim 1, wherein R1 is selected from the group consisting of glycine, alanine, valine, leucine, isoleucine, glutamine, glutamic acid, asparagine, aspartic acid, cysteine, methionine, serine, and threonine.

7. A propofol derivative comprising the formula
(X—Yₘ)ₙ—S,
wherein X has the formula: 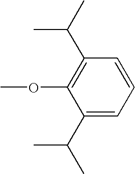
Y is a bifunctional linker,
S is a poly- or oligosaccharide moiety,
n is equal or less than the number of the terminal saccharide units in the poly- or oligosaccharide S, and
m is, independent of n, 0 or 1.

8. The propofol derivative of claim 7, wherein m=0 and propofol and S are linked to each other by an ester bond consisting of an oxygen of X and a terminal carbonyl derivative of S.

9. The propofol derivative of claim 7, wherein m=1 and propofol and S are linked to each other by means of a bifunctional linker Y, said bifunctional linker Y preferably being linked to propofol by an ester, carbonate or carbamate bond and being linked to S by an amide, imine, secondary amine, ester, thioester, carbonate, carbamate, urea or disulfide bond.

10. The propofol derivative of claim 7, wherein S is an oligosaccharide comprising at most 1 to 20, preferably 1 to 10, more preferably 2 to 7 saccharide units.

11. The propofol derivative of claim 7, wherein S is a polysaccharide comprising more than 20 saccharide units, preferably 20 to 100, more preferably 20 to 50 saccharide units.

12. The propofol derivative of claim 7, wherein the poly- or oligosaccharide S is linear and the saccharide units are linked by α(1-4) bonds.

13. The propofol derivative of claim 7, wherein at least one terminal saccharide unit of S is derived from an aldose monosaccharide comprising a free aldehyde group.

14. The propofol derivative of claim 7, wherein the viscosity of said compound is 1-100 mPasc, preferably 1-20 mPasc, more preferably 1-7 mPasc.

15. The propofol derivative of claim 7, wherein the molar ratio of propofol to S is in the range of 10:1 to 1: 1, preferably in the range of 5:1 to 1:1, and most preferably about 1:1.

16. The propofol derivative of claim 7, wherein S comprises one or more of the poly- or oligosaccharide unit(s) selected from the group consisting of:
a) monosaccharides, preferably: ribose, arabinose, xylose, lyxose, allose, altrose, glucose, mannose, gulose, idose, galactose, talose, fucose;
b) disaccharides, preferably lactose, maltose, isomaltose, cellobiose, gentiobiose, melibiose, primeverose, rutinose;
c) disaccharide homologues, preferably maltotriose, isomaltotriose, maltotetraose, isomaltotetraose, maltopentaose, maltohexaose, maltoheptaose, lactotriose, lactotetraose;
d) uronic acids, preferably glucuronic acid, galacturonic acid;
e) branched oligosaccharides, preferably panose, isopanose,
f) amino monosaccharides, preferably galactosamine, glucosamine, mannosamine, fucosamine, quinovosamine, neuraminic acid, muramic acid;, lactosediamine, acosamine, bacillosamine, daunosamine, desosamine, forosamine, garosamine, kanosamine, kansosamine, mycaminose, mycosamine, perosamine, pneumosamine, purpurosamine, rhodosamine;
g) modified saccharides, preferably abequose, amicetose, arcanose, ascarylose, boivinose, chacotriose, chalcose, cladinose, colitose, cymarose, 2-deoxyribose, 2-deoxyglucose, diginose, digitalose, digitoxose, evalose, evemitrose, hamamelose, manninotriose, melibiose, mycarose, mycinose, nigerose, noviose, oleandrose, paratose, rhodinose, rutinose,sarmentose, sedoheptulose, solatriose, sophorose, streptose, turanose, tyvelose.

17. The propofol derivative of claim 15, wherein S comprises one or more of the saccharide unit(s) selected from the group consisting of glucosamine, galactosamine, glucuronic acid, galacturonic acid, lactose, lactotetraose, maltose, maltotriose, maltotetraose, isomaltose, isomaltotriose, isomaltotetraose, and neuraminic acid.

18. The propofol derivative of claim 7, wherein the bifunctional linker Y comprises a linear or branched aliphatic chain, preferably an aliphatic chain of 1 to 20, more preferably 1 to 12, most preferably 2 to 6 carbons.

19. The propofol derivative according to claim 7, wherein the bifunctional linker Y is
—HN—(CH₂)ₓ—NH—CO—(CH₂)_{y}—CO—,
wherein x=0 to 10, preferably x=0, and y=1 to 5, preferably y=1 or 2.

20. The propofol derivative according to claim 7, wherein S is a monosaccharide, disaccharide, oligosaccharide or polysaccharide and comprises at least one saccharide unit selected from the group consisting of allose, altrose, glucose, mannose, gulose, idose, galactose, talose, sucrose, lactose, maltose, isomaltose, cellobiose, maltobionic acid, and lactobionic acid.

21. The propofol derivative according to claim 7, wherein S is maltotrionic acid, lactobionic acid or hydroxyethyl starch.

22. The propofol derivative according to claim 7, wherein S comprises at least 2 hydroxyethyl glucose units, wherein the hydroxy ethyl glucose units may be furtner substituted.

23. A process for preparing propofol derivatives according to claim 7, comprising the steps of:
a) coupling propofol with one or more terminal aldehyde group(s) of a poly- or oligosaccharide S, or
b) coupling propofol with one or more terminal carboxylic group(s) of a poly- or oligosaccharide S, or
c) coupling propofol with one or more activated terminal carboxylic group(s) of a poly- or oligosaccharide S.

24. The process of claim 23, further comprising a step b′) or c′) prior to step b) or c), respectively, wherein one or more terminal aldehyde group(s) of a poly- or oligosaccharide S precursor are selectively oxidized to produce the poly- or oligosaccharide S.

25. The process of claim 24, wherein the one or more terminal aldehyde group(s) of poly- or oligosaccharide S are selectively oxidized to carboxylic acid group(s) or activated carboxylic acid group(s) using
(i) halogen, preferably I₂, Br₂, in alkaline solution, or
(ii) metal ions, preferably Cu⁺⁺ or Ag⁺, in alkaline solution, or
(iii) by electrochemical oxidation.

26. The process of claim 23, wherein in step c) the one or more activated terminal carboxylic group(s) of a poly- or oligosaccharide S are selected from the group consisting of a lactone, an anhydride, a mixed anhydride, and halogenide of a carboxylic acid.

27. The process of claim 26, wherein in step c) the one or more activated terminal carboxylic group(s) of a poly- or oligosaccharide S is (are) a lactone group(s).

28. A process for preparing propofol derivatives according to claim 1, comprising the steps of:
a) coupling a suitable bifunctional linker group(s) Y to propofol, and
b) coupling the product(s) of step a) with one or more terminal aldehyde, carboxylic acid, or activated carboxylic group(s) of a poly- or oligosaccharide S, or
a′) coupling a suitable bifunctional linker group(s) to one or more terminal aldehyde, carboxylic acid, or activated carboxylic group(s) of a saccharide S, and
b′) coupling the product(s) of step a) with one or more propofol.

29. A process according to claim 28, wherein an imine bond that is formed between the bifunctional linker group and the component S is further reduced to a secondary amine.

30. The process of claim 29, wherein the imine is reduced by NaBH₃CN at pH values of 6-7.

31. The process of claim 28, wherein in step b) or step a′) the one or more activated terminal carboxylic group(s) of a poly- or oligosaccharide saccharide S selected from the group consisting of a lactone, an anhydride, a mixed anhydride, and a halogenide of a carboxylic acid.

32. The process of claim 31, wherein the coupling of a lactone poly- or oligosaccharide derivative S and one or more bifunctional linkers Y is performed in the absence of an activator.

33. The process of claim 32, wherein the lactone is coupled in non-protic solvents, preferably DMF, DMSO, N-methylpyrrolidone, or alcohols, preferably, MeOH, EtOH, n-PrOH, i-PrOH, n-butanol, iso-butanol, tert-butanol, glycol or glycerol.

34. The process of claim 28, wherein the bifunctional linker comprises an aliphatic chain of 1 to 20, more preferably 1 to 12, most preferably 2 to 6 carbon atoms.

35. The process of claim 28, wherein the bifunctional linker is a linker that has an amino functional group on one side to be coupled to the terminal saccharide moiety of S and an activated carboxylic function at the side to be coupled to propofol.

36. The process of claim 28, wherein the bifunctional linker is
—HN—(CH₂)_{X}—NH—CO—(CH₂)_{y}—CO—,
wherein X=0 to 10, preferably X=0, and Y=0 to 5, preferably Y=1 or 2.

37. A method for anesthetizing a mammal, wherein a therapeutically effective amount of a compound according to claim 1 is administered to said mammal.

38. A method of treating convulsions or migraine or for inhibiting free radicals in a mammal, wherein a therapeutically effective amount of a propofol derivative according to claim 1 is administered to said mammal.

39. A propofol derivative according to claim 1 for use as a medicament.

40. Use of a propofol derivative according to claim 1 for the preparation of a medicament for anesthetizing a mammal.

41. Use of a propofol derivative according to claim 1 for the preparation of a medicament for treating and/or preventing convulsions, migraine or for inhibiting free radicals in a mammal.

42. A pharmaceutical composition comprising the propofol derivative of claim 1 and a pharmaceutically acceptable carrier, more preferably comprising an α-proline propofol ester and a pharmaceutically acceptable carrier.

43. A kit comprising the propofol derivative of claim 1 in a dehydrated form, preferably in lyophilized form, and at least one physiologically acceptable aqueous solvent.
